(19) United States
(12) Patent Application Publication
(10) Pub. No.: US 2017/0013785 A1
(43) Pub. Date: Jan. 19, 2017

(54) ENGRAFTED PLANTS HAVING AT LEAST ONE ROOTSTOCK WITH PREDETERMINED GROWTH CONTROL PERIOD

(71) Applicant: VIRENTES LTD., M.P. Misgav (IL)

(72) Inventor: Yuval Fradkin, Tel Adashim (IL)

(21) Appl. No.: 15/123,910

(22) PCT Filed: Mar. 9, 2015
(86) PCT No.: PCT/IL2015/050245

§ 371 Date: Sep. 6, 2016

Related U.S. Application Data
(60) Provisional application No. 61/950,110, filed on Mar. 9, 2014.

(51) Int. Cl. A01G 1/06; A01H 5/00; A01G 1/00

(52) U.S. Cl. CPC A01G 1/06; A01G 1/001; A01H 5/00

(57) ABSTRACT

The present invention provides a plant grafting method using a plurality of rootstocks grafted together to one scion. A collar is positioned around at least one rootstock to limit a period of its contribution for growth and development of said plant. The grafting method benefits from simultaneous utilization of plurality of rootstocks with versatile growth control.

### FIELD OF THE INVENTION

The present invention relates to a plant grafting method. In particular, the present invention relates to methods for grafting a scion with a plurality of rootstocks, providing differential growth control.

### BACKGROUND OF THE INVENTION

Plant grafting is a widely used technique in agriculture that includes the insertion or attachment of one plant part to another so that the two sets of vascular tissues may join together. Callous tissue forms at the graft union as part of the normal healing process of the plant and serves as a conduit for water and nutrients between the plant parts. In most cases, one plant contributes the bottom part that includes roots and is called the stock or rootstock. The other plant is usually selected for its stems, leaves, flowers, or fruits and this upper part is called the scion. Rootstocks are selected to improve soil compatibility and rooting, to provide resistance to soil-borne pests and diseases, to increase tolerance to environmental stresses and to the overall improvement of crop productivity and/or quality. Usually the grafting method comprises a single rootstock and a single scion, utilizing the advantages of both plant parts.

Many types of grafting methods were described over the years. For example:

U.S. Pat. No. 4,012,866 discloses a method for asexual propagation of a difficult-to-root plant species, such as an avocado tree. The method provides the positioning of a collar loosely around a rootstock grown from a bud grafted to a seedling, wherein the bud is from a clone having a desired disease immunity and growth characteristics. After the rootstock has grown to a given size, a second grafting of a fruiting scion may be made. Then, the initial seedling, root stock, collar and fruiting scion are transplanted as a body. The rootstock grows within the collar causing the collar to gradually constrict the flow of nutrients to the seedling and simultaneously promote rooting of the rootstock itself, as the seedling is killed off. The seedling itself finally disintegrates, leaving a properly rooted plant having the desired characteristics.

U.S. Pat. No. 4,937,971 discloses the process of concomitantly rooting and uniting plants which are in an active growing state. The process comprises herbaceous cutting, assembling of a single bud scion to the top of another scion, and planting the graft unit in a rooting medium in the appropriate environment until the desired stage of growth. The invention mainly refers to the grafting of grape vine saplings.

U.S. Pat. No. 6,901,696 discloses a grafting method comprising the preparation of stool followed by root and scion grafting into the stool. The stool is then placed into a rooting medium.

U.S. Pat. No. 8,186,099 discloses methods for obtaining double trunk plants. The methods include a single grafting at different growth stages of trees followed by shoot selection to establish two trunks plants. Double trunk trees provide improved photosynthesis effectiveness, higher fruit size and color homogeneity, better controlling of plant strength and flower induction.

Different means of cutting and joining of scion to rootstock were described over the years. One example is the “approach graft” that teaches the method of growing of rootstock and scion sources close to each other, slicing both stems at a convenient height above soil and then joining the stems. After a certain healing time, the top part of the stock plant and the root part of the designated scion are cut back.

As described above, rootstocks are selected to overcome variety of environmental stresses and problems. However, rootstocks may have negative effects upon the scion phenotype, particularly in herbaceous seedlings. For example, a rootstock of a gourd may confer several unfavorable traits to a watermelon scion such as stiffed texture and decreased sugar level. In other examples, the rootstock may cause dwarfing diseases, or increase the size of the plants, having deleterious effects on the yield. The negative effects, in many cases, are reflected in certain periods throughout the plant growth.

Therefore, there is a need for developing additional means of grafting, particularly for herbaceous crops providing the enablement of a differential growth control.

### SUMMARY OF THE INVENTION

The present invention provides a grafting method comprising at least one rootstock exerting a limited or predetermined period of contribution to growth and development.

According to one aspect the present invention provides a grafting method using two or more rootstocks simultaneously that optimizes the contribution of the individual rootstock to the grafted plant. Thus there is provided, in accordance with some embodiments, an engrafted plant, said engrafted plant comprises a scion and two assembled rootstocks, wherein the growth of one is controlled by a collar. When the rootstock with the collar grows in diameter, the contact between the collar and the rootstock gradually impairs the plant transport system. In some embodiments, the process leads to a growth arrest of the rootstock during a predetermined growth stage and finally to the rootstock death. In other embodiments, the collar constriction leads to partial degeneration or growth limitation. The second rootstock that is free of said collar continues to grow and to support the scion. The rootstock with the collar may contribute to the traits of the engrafted plant mainly through the first stages of the plant development. According to some embodiments, the second rootstock forms part of the final plant, providing for its compatibility with the soil growth environment. In some embodiments, the rootstocks possess a synergism effect.

An advantage of the present invention over hitherto known grafting methods is the use of a combination of a plurality of rootstocks that exerts its effect in a controlled manner and for predetermined period. Unlike the “approach graft” described hereinabove, which requires additional cutting step following the healing period, the method of the present invention provides for a single grafting procedure. In addition, the grafted scion(s) benefits from the influence of the plurality of rootstocks from immediately after grafting and up to a predetermined, desired time point. According to the teachings of the present invention, the collar renders the second cutting step of removing one of the rootstocks unnecessary; the collar implements a gradual disconnection of the designated rootstock, which may confer less stress than cutting. Moreover, the collar enables controlling the extent of effect the designated rootstock applies to the scion.

According to a first aspect, the present invention provides a method for producing an engrafted plant, the method comprising: (i) providing a plurality of rootstocks; (ii) positioning a collar around at least one of the rootstocks; and (iii) grafting the plurality of rootstocks with at least one scion; to obtain an engrafted plant having a plurality of rootstocks each supporting growth for a predetermined period of growth.

The rootstock on which the collar is positioned may support the scion throughout the engrafted plant growth period or at first stages of the growth period. According to certain embodiments, the rootstock around which the collar is positioned supports the scion throughout the growth period, wherein said rootstock supports the scion at differential intensities during the development of the engrafted plant. According to certain embodiments, the collar arrests the growth of the rootstock comprising same and therefore said rootstock is temporal. According to some embodiments, the collar arrests the growth of the rootstock comprising same at a predetermined developmental stage. According to certain exemplary embodiment, following the growth arrest of the temporal rootstock, the remaining rootstock or rootstocks maintain the grafted scion.

According to certain embodiments, the collar is made of a rigid material. According to certain embodiments, the rigid material is selected from the group consisting of metal, plastic, and a combination thereof. Each possibility represents a separate embodiment of the invention. According to other embodiments, the collar is made of a flexible material that is stretched against the growing rootstock, thus controls the growth of said rootstock. According to exemplary embodiments, the flexible material is a rubber.

According to certain embodiments, the collar further comprises at least one growth inhibitor. According to certain embodiments, the growth inhibitor is an herbicide. According to an exemplary embodiment, the growth inhibitor is Glyphosate (N-(phosphonomethyl)glycine). In other exemplary embodiments, the herbicide is Paclobutrazol (α-tert-Butyl-β-(4-chlorobenzyl)-1H-1,2,4-triazole-1-ethanol). It will be apparent to one of skill in the art that the herbicide should act locally on the rootstock to which the collar is positioned, to avoid action on the rest of the plant. In alternative embodiments the rest of the plant must be resistant to the herbicide placed in the collar. According to other embodiments, the growth inhibitor is a hormone.

According to certain embodiments, at least two collars are positioned around the rootstock, wherein each of the collars affects the rootstock at a different intensity.

According to certain embodiments, at least one collar is positioned around each of the rootstocks. According to some embodiments, each of the collars affects the rootstocks at different intensity.

According to certain embodiments, the source of the rootstock around which the collar is positioned is a vigorous plant such that said rootstock impacting the first stage of growth of the engrafted plant and the additional rootstock supports the growth and development of the engrafted plant throughout the entire growth period.

The rootstocks and the scion can be members of different taxonomy origins, with the stipulation that they are compatible for grafting with each other.

According to certain embodiments, the plurality of rootstocks is of plants of the same family.

According to certain embodiments, the rootstocks and the scions are of herbaceous plants.

According to certain exemplary embodiments, the rootstocks and the scion are of the Cucurbitaceae family. According to some embodiments, one of the rootstock is of a watermelon source and the second is of a gourd source. In an exemplary embodiment, the method is used for grafting a watermelon, wherein the temporal rootstock on which the collar is positioned is a gourd. According to some embodiments, the gourd contributes to the vigor of the grafted plant.

According to certain embodiments, the plurality of rootstocks and the scion are of the solanaceae family.

According to certain embodiments, the plurality of rootstocks is of a pepper (Capsicum) or tomato (Solanum lycopersicum) source. Each possibility represents a separate embodiment of the invention.

According to certain embodiments, the plants, which are the source for the rootstocks, are seeded and grown next to each other. According to other embodiments, said plants are grown separately and later transplanted next to each other. According to certain embodiments, said plants are grown in the same plant tray. According to other embodiments, said plants are planted in different plant trays.

According to an additional aspect, the present invention provides a method for producing an engrafted plant, the method comprising: (i) providing at least one rootstock and an intact plant; (ii) positioning a collar around the at least one rootstock; and (iii) grafting the rootstock to the intact plant; to obtain an engrafted plant having a plurality of rootstocks each supporting growth for a predetermined period of growth.

The rootstock source as well as the collar type is as described hereinabove.

The rootstock may support the plant for a partial or entire growth period, at the same or or at different intensities as described hereinabove.

According to certain embodiments, the plant is herbaceous plant. According to some embodiments, the plant is of the Cucurbitaceae family. According to exemplary embodiments, the plant is a watermelon. According to additional embodiment the rootstock is of a gourd source. According to other embodiments, the plant is of the solanaceae family.

According to some embodiments, the intact plant and the at least one rootstock are of herbaceous plants.

According to another aspect, the present invention provides an engrafted plant comprising a scion and a plurality of rootstocks, wherein a collar is positioned around at least one of said rootstocks.

According to certain embodiments, the rootstocks, the scion and the collar are as described hereinabove.

According to some embodiments, the engrafted plant is herbaceous plant. According to certain embodiments, the engrafted plant is of the Cucurbitaceae family. According to exemplary embodiment, the scion and one of the rootstocks are of a watermelon plant and the second rootstock is of a gourd. According to other embodiments, the scion and the rootstock are of a pepper or tomato source.

According to certain embodiments, the collar is as described hereinabove.

According to a yet another aspect, the present invention provides an engrafted plant comprising at least one rootstock grafted to an intact plant, wherein a collar is positioned around the at least one rootstock.

According to certain embodiments, the plant and the at least one rootstock as well as the collar are as described hereinabove.

According to certain embodiments, the plant and the at least one rootstock are of herbaceous plants.

According to additional aspect, the present invention provides a method for producing an engrafted herbaceous plant, the method comprising: (i) providing at least one rootstock; (ii) positioning a collar around the at least one rootstock; and (iii) grafting the at least one rootstock with at least one scion.

According to certain embodiments, the scion is supported by a single rootstock. According to these exemplary embodiments, the rootstock supports the scion at differential intensities throughout the entire plant growth.

According to certain embodiments, the at least one scion and the at least one rootstock are of herbaceous plants.

Other objects, features and advantages of the present invention will become clear from the following description and drawings.

### BRIEF DESCRIPTION OF THE FIGURES

FIG. 1 depicts the grafting method steps of two rootstocks and a scion according to one of the embodiments of the invention. One of the rootstocks has a collar positioned around it. The scion is grafted onto the upper side of one of the rootstocks.

FIG. 2 depicts the grafting method steps of two rootstocks and a scion according to additional embodiment of the invention. A collar is positioned around one of the rootstocks. The scion is grafted onto the upper side of both rootstocks.

FIG. 3 depicts an example of grafting a rootstock and an intact plant according to another aspect of the invention. A collar is positioned around the rootstock. The rootstock is grafted with the intact plant.

FIG. 4 depicts different options of grafting two scions onto two rootstocks according to the invention. FIG. 4a depicts an engrafted plant wherein the two scions are attached to same joining point. FIG. 4b depicts an engrafted plant wherein the two scions are attached on top of the other.

FIGS. 5a-5b shows engrafted tomato plant comprising a scion grafted onto tomato rootstocks. FIG. 5a shows the engrafted plant immediately after the grafting procedure. FIG. 5b shows older engrafted plant. The grafting point was covered with Teflon and the engrafted plants were planted in the field at 80 cm intervals.

FIG. 6 shows a pepper rootstock grafted to an intact pepper seedling. The connecting point, which was fixed with grafting clips, is enlarged.

FIG. 7 shows an engrafted plant according to the invention in a field trial. The engrafted plant includes a scion of a watermelon and a rootstock of a gourd, wherein a plastic collar (marked with an arrow) is positioned around the gourd rootstock. The collar is designed to suffocate and degenerate the gourd rootstock in a later developmental stage.

### DETAILED DESCRIPTION OF THE INVENTION

The present invention provides grafting methods for improving plant traits. According to certain embodiments, the present invention discloses the use of at least two joined rootstocks, wherein one rootstock degenerates at a predetermined growth stage by a collar positioned around it. The grafting configuration further comprises a scion conferring desired fruit and/or flower traits. The engrafted plant according to the present invention benefits from the combination of two or more rootstocks that contributed to the growth of the engrafted plant at different growth stages and/or periods. In some embodiments, the rootstocks possess a synergism effect in terms of total vegetative growth, crop yield or crop quality. For example, one rootstock may affect the color of the fruit and the other its sugar content resulting in higher quality fruit. In another example, both rootstocks are needed to overcome soil-borne pests and/or diseases. The methods of the present invention are particularly advantageous when a rootstock that supplies vigor, which is required mostly through the first growth stages, confers negative effect(s) at later growth stages. The collar positioned around such rootstock arrest its growth at a designated time (determined by the power enforced by the collar on the rootstock). The rootstock(s) free of the collar provides for beneficial traits in all or later growth stages of the engrafted plant, including, for example, enhanced resistance to diseases and/or abiotic stresses.

### Definitions

The terms “grafting” or “engrafting” (in any tense used) are used herein interchangeably and refer to the uniting of at least two parts of growing plants such that the two sets of vascular tissues may join together by insertion or by placing in close contact. Typically grafting comprises the point of insertion of a scion upon a rootstock. However, any part of two plants which are unnaturally assembled is called grafting. Grafting is used in its broadest scope and includes budding as well. In budding, single bud from the desired scion is used to be joined with the rootstock rather than an entire twig.

The term “rootstock” refers to the lower part of a grafted plant that supply the roots. Usually the rootstock is selected for its superior qualities such as disease resistance, vigor, ability to grow in adverse soil conditions, and additional impacts on the scion growth. The term “rootstock”, according to the present invention, relates also to the lower part of a plant once another rootstock is grafted to it.

The term “temporal rootstock” as used herein refers to a rootstock that cease from supporting the growth of an engrafted plant comprising same after a certain period of time.

The terms “engrafted plant” and “grafted plant” in single or plural forms are used herein interchangeably and refer to a plant composed of a scion joined with at least one rootstock by grafting.

The term “plurality” as used herein refers to at least two.

The term “scion” refers to the upper part of a grafted plant which is a detached section of a shoot or a twig. The scion is joined to the lower part which is called a rootstock. Usually the scion is selected for superior fruiting or flowering traits. The scion, according to the present invention, may be also a bud. The term “bud” refers to a scion having a single axillary bud eye.

The term “the same taxonomy origin” is used herein in its broadest scope and refers to plants of the same family, genus, or species. Usually, grafted plants should be of the same family; however, plants from different families may be also grafted together according to this invention.

The terms “differential intensity”, “differential intensities” and “differential growth control” are used herein interchangeably with regard to a diverse degree of effect and/or influence of a rootstock on a grafted scion and/or engrafted plant exerted along the growth period of the engrafted plant. The collar of the invention may partially suffocate the rootstock as to enable only partial influence (i.e., lower intensity) of a rootstock with a collar as compared to same rootstock without the collar. In other embodiments, the collar is designed to completely suffocate the rootstock. In these cases, the rootstock influences the scion and/or engrafted plant at different intensities for the first growth stages, and then the rootstock degenerates and dies.

The term “predetermined period growth” refers to the time a rootstock supports the scion which can be until a specific stage of development or throughout the entire growth of the plant.

The term “stretched against” is used herein with regard to the force conferred by a collar on a rootstock around which it is positioned. As the rootstock perimeter grows, the collar increases the pressure upon the rootstock, resulting in reducing its influence upon the scion.

The dimensions of the collar are readily determined by one of skilled in the art according to plant species or variety, the developmental stage in which the growth is to be controlled and the required degree of growth control. According to certain embodiments, the diameter of the collar is about 20%, 30%, 40%, 50%, 60%, 70%, 80% or 90% of the stem diameter of a unencumbered mature plant from which the rootstock is derived. According to certain embodiments, the diameter of the collar is about 20% to about 90% of the stem diameter of an unencumbered mature plant from which the rootstock is derived. According to certain embodiments, the diameter of the collar is about 35% to about 75% of the stem diameter of an unencumbered mature plant from which the rootstock is derived. According to certain embodiments, the diameter of the collar is about 20% to about 40% of the stem diameter of an unencumbered mature plant from which the rootstock is derived. According to certain embodiments, the diameter of the collar is about 40% to about 60% of the stem diameter of an unencumbered mature plant from which the rootstock is derived. According to certain embodiments, the diameter of the collar is about 60% to about 80% of the stem diameter of an unencumbered mature plant from which the rootstock is derived. According to certain embodiments, the diameter of the collar is about 70% to about 90% of the stem diameter of an unencumbered mature plant from which the rootstock is derived. Each possibility represents a separate embodiment of the present invention.

As used herein the term “about” in reference to a numerical value stated herein is to be understood as the stated value +/−10%, more preferably +/−5%, even more preferably +/−1%, and still more preferably +/−0.1% from the specified value, as such variations are appropriate to achieve the intended purpose.

According to some embodiments, the collar is designed to gradually suffocate the rootstocks before and/or during the ripening of the fruits. Without wishing to be bound by any specific theory or mechanism of action, the collar confers differential degeneration of the rootstock which reduces the vigorous effect of the rootstock upon the scion with fewer traumas to the plant.

Reducing irrigation during ripening in crops, such as tomatoes, is a known practice in agriculture. The reduction in water may increase the sugar contents and improve pest resistance. Partial suffocation of the rootstock by the collar may confer a similar effect.

According to certain exemplary embodiments, the diameter of the collar is designed as to arrest the growth of the rootstock such that the rootstock bearing the collar degenerates. According to these embodiments, the collar diameter is up to about 20%, 30%, 40%, 50%, 60%, or 70% of the diameter of a mature plant from which the rootstock is derived. Each possibility represents a separate embodiment of the invention.

According to additional exemplary embodiments, the diameter of the collar is designed as to control the rootstock influence on the scion. According to these embodiments, the collar diameter is at least 70%, 80%, 90% or more the diameter of a mature plant from which the rootstock is derived. Each possibility represents a separate embodiment of the invention.

The term “intact plant” refers to a plant having both the upper part, i.e. including a stem and leaves; and the bottom part which includes roots. In contrary to a rootstocks or scion, the intact plant is continuous from roots to shoots.

According to one aspect, the present invention provides a method for producing an engrafted plant, the method comprising: (i) providing a plurality of rootstocks; (ii) positioning a collar around at least one of the rootstocks; and (iii) grafting the plurality of rootstocks with at least one scion; to obtain an engrafted plant having a plurality of rootstocks providing differential growth control.

According to certain embodiments, the present invention provides a method for producing an engrafted plant, the method comprising: (i) providing two rootstocks; (ii) positioning a collar around one of the rootstocks; and (iii) grafting the rootstocks together with one scion; to obtain an engrafted plant having a plurality of rootstocks each supporting growth for a predetermined period of growth.

According to certain embodiments, the collar arrests the growth of the rootstock in a predetermined growth stage, and therefore said rootstock is temporal. According to exemplary embodiments, following the growth arrest of the temporal rootstock, the remaining rootstock(s) maintains the grafted scion forming together the engrafted plant.

According to certain embodiments, the collar partially damages the rootstock, limiting its growth. According to these exemplary embodiments, the rootstock with the collar has reduced influence on the scion as compared to the same rootstock without said collar.

More than one collar may be positioned around a single rootstock, controlling its growth in different and predetermined developmental stages. The collar can be at any shape with the stipulation that it surrounds, at least partially, the rootstock's stem, enabling the growth control according to the present invention.

According to certain embodiments, the collar is made of a rigid material. According to certain embodiments, the rigid material is selected from the group consisting of metal, plastic, and a combination thereof. Each possibility represents a separate embodiment of the invention. According to other embodiments, the collar is made of a flexible material that is stretched against the growing temporal rootstock, thus controlling the growth of said rootstock. According to certain embodiments, the flexible material is a rubber.

According to certain embodiments, the collar further comprises at least one growth inhibitor. According to certain embodiments, the growth inhibitor is an herbicide. According to other embodiments, the growth inhibitor is a hormone.

The collar may be positioned at any height of the rootstock, with the stipulation that at the designated growth stage it will degenerate and/or suffocate the stem, partially or completely. In one exemplary embodiment, the collar is positioned at about two thirds of the rootstock height. According to other embodiments, the collar is positioned at the ground level.

According to exemplary embodiments, the collar comprises an additional chemical that affects the roots. In some embodiments, the collar further comprises fertilizers.

The collar diameter depends on the plant type, its age and the purposes of the collar. According to certain embodiments the collar diameter is so designed as to enable expansion of the rootstock diameter until a predetermined value, thereafter the force applied by the collar on the rootstock results in deterioration of the rootstock leading to growth arrest and optionally death of the rootstock. According to other embodiments, the force applied by the collar on the rootstock results in decreasing the influence of the rootstock upon the scion. According to certain exemplary embodiments, the collar diameter is from about 3 mm to about 20 mm. According to certain embodiments, the collar diameter is from about 5 mm to about 17 mm. According to certain embodiments, the collar diameter is from about 7 mm to about 15 mm. According to certain embodiments, the collar diameter is from about 9 mm to about 14 mm. According to additional exemplary embodiments, the collar diameter is about 3, 5, 7, 10, 13, 17, or 20 mm. Each possibility represents a separate embodiment of the present invention.

The assembling angles as well as the cutting of the rootstocks and the scions are performed by any grafting means with the stipulation that the vascular systems of the different parts will be in contact. The term “assembling angles” refers to the angle between to grafted parts of the engrafted plant according to the invention.

According to certain embodiments, the temporal rootstock is of a vigorous plant, impacting the first stage of growth of the grafted plant and the additional rootstock supports the growth and development of the grafted plant through the entire growth period.

There may be more than two rootstocks. According to certain embodiments, three rootstocks are grafted with a single scion wherein at least one rootstock has a collar. Each rootstock contributes certain desired traits, that may be the same or different for each rootstock. This grafting configuration enables a sequential growth control wherein each rootstock influences the scion for a predefined period of time determined by the collar configuration and resulting in a controlled effect. According to certain embodiments, four rootstocks are grafted with a single scion, wherein at least one rootstock has a collar. According to certain embodiments, the engrafted plant benefits from the synergism of several rootstocks for a predetermined periods of time.

The rootstocks and the scions can be of different taxonomy origin with the stipulation that they are compatible for grafting with each other. Typically, the rootstock and the scion candidates are selected from the same family. According to certain embodiments, the rootstocks are of different species or strains.

According to certain embodiments, the rootstocks and the scions are of herbaceous plants.

According to certain exemplary embodiments, the rootstocks and the scion(s) are of the Cucurbitaceae family. In an exemplary embodiment, the method is used for grafting a watermelon, wherein the temporal rootstock is of a gourd. According to one embodiment, the gourd contributes to the vigor of the grafted plant. According to this exemplary embodiment, said gourd rootstock confers negative effects upon the ripening phase.

The mini watermelon (Citrullus lanatus) ‘ecstazy’ variety is susceptible to several soil pathogens such as Monosporascus cannonballus, Macrophomina phasoliona and different species of Fusarium. These pathogens generate diseases that result in rotting and wilting of the watermelon roots and stem. The hitherto solution is the grafting of the mini watermelon scion with a gourd rootstock that provides vigor and resistance to the soil pathogens. However, the grafting of the gourd rootstock results in several unfavorable traits, including bigger-size watermelons (which are less favorable in the market), bright color tissue, stiffed texture, decreased sugar level, and unfavorable taste. According to certain embodiments, the present invention provides a grafting configuration that comprises the mini watermelon scion and two rootstocks. One rootstock is of a watermelon that is partially resistant to Fusarium. The second rootstock is of a gourd designated to provide vigor and resistance during the first developmental stages. The gourd rootstock has a collar positioned around it such that the collar arrests growth of the gourd rootstock at a predetermined growth stage. As a result, the gourd rootstock confers its beneficial traits on the engrafted plant without conferring the undesirable effects during the ripening stages. After the gourd growth is arrested or completely stopped, the watermelon remaining rootstock maintains the mini watermelon scion and support growth of the engrafted plant.

According to certain embodiments, the rootstock and the scion are derived from plants of the solanaceae family

According to other exemplary embodiments, the present invention provides a method for grafting a tomato or pepper plant. Unexpectedly, grafting a single scion onto two rootstocks results in an increase of the fruit size as well as the total fruit yield. Fitting a collar around at least one of the rootstocks allows a control of its contribution over the developmental stages.

According to certain embodiments, the scion/scions and at least one of the rootstocks are from the same source. The term “same source” as used herein may be, for non-limiting examples, the same family, species, variety or plant.

According to certain embodiments, the plurality of rootstocks is from the same species. According to some embodiments, synergism of the two or more rootstocks confers the desired traits.

The method of the present invention may be employed to any plant. According to certain embodiments, the method is employed for grafting trees. According to other embodiments, the method is employed for grafting vegetables. According to a yet other embodiments, the method is employed for grafting ornamental plants.

According to certain embodiments, the plants that are used as a source for the rootstocks are seeded and grown next to each other. According to other embodiments, said plants are grown separately and later are transplanted next to each other. According to certain embodiments, said plants are grown in the same plant tray. According to other embodiments, said plants are planted in different plant trays.

According to certain embodiments, the method according to the present invention is applied for grafting two scions onto one or plurality of rootstocks. Without wishing to be limited, grafting of two scions is typically used for ornamental plants. According to certain embodiments, the two scions are grafted onto the top of at least one rootstock. According to other embodiments, one scion is grafted onto the top of another scion and then the lower scion is grafted onto the top of at least one rootstock.

According to certain embodiments, the grafting configuration comprises two rootstocks and two scions wherein one of the rootstocks has a collar positioned around it. According to other embodiments, the grafting configuration comprises three rootstock and two scions, wherein at least one of the rootstocks has a collar positioned around it.

The two (or more) sources for the rootstocks should be planted in a convenience distance from each other such that, on one hand will enable the grafting and on the other hand will keep enough space for root development. The distance between the rootstocks depends on the plant type and the developmental stage. According to exemplary embodiments, the planting distance of the rootstocks of Cucurbitaceae is from about 5 cm to about 20 cm

The scion and the plurality of rootstocks may be grafted at the same time or sequentially. In a complicated grafting procedure, one can separate the grafting steps, allowing healing periods between the steps as known for a skilled artisan. According to certain embodiments, the plurality of rootstocks are grafted to the at least one scion at the same grafting procedure. According to other embodiments, the grafting performed in more than one step.

According to additional aspect, the present invention provides a method for producing an engrafted plant, the method comprising: (i) providing at least one rootstock; (ii) positioning a collar around the at least one rootstock; and (iii) grafting the at least one rootstock with at least one scion.

According to additional aspect, the present invention provides a method for producing an engrafted plant, the method comprising: (i) providing at least one rootstock and at least one scion; (ii) positioning a collar around at least one scion; and (iii) grafting the at least one rootstock with the at least one scion.

According to additional aspect, the present invention provides a method for producing an engrafted plant, the method comprising: (i) providing at least one rootstock and at least one scion; (ii) positioning a collar around the at least one rootstock and/or at least one scion; and (iii) grafting the at least one rootstock with the at least one scion.

According to certain embodiments, the scion is supported by a single rootstock. According to these exemplary embodiments, the rootstock supports the scion at differential intensities throughout the entire plant growth.

According to some embodiments, the collar is positioned above or below the grafting point.

Without wishing to be bound by any specific theory or mechanism of action, the rootstock confers negative genetic and/or physiological traits upon the scion. These effects may be derived from excess water and/or solutes that are transferred through the vascular tissue. In some embodiments, the collar limits the transfer of the water and/or solutes.

The collar may also prevent downward flow of organic nutrients such as sucrose, from the upper part of the plant toward the plant roots. Decreasing transport of photosynthesis products may result in yield improvement. According to some embodiments, the collar is positioned around at least one of the rootstocks. According to certain embodiments, the collar is positioned around the scion.

According to certain examples, the collar is positioned around any height of the scion. According to some exemplary embodiments, the collar partially suffocate the scion, preventing downward of organic nutrients from the scion to the rootstocks. According to other exemplary embodiments, the collar reduces negative effects conferred by the rootstock upon the scion.

According to some embodiments, the collar diameter is at least 70%, 80%, 90%, 95% or more the diameter of a mature plant from which the scion is derived. Each possibility represents a separate embodiment of the invention. According to certain embodiments, the collar diameter is from about 60% to about 80% of the diameter of a mature plant from which the scion is derived. According to certain embodiments, the collar diameter is from about 70% to about 90% of the diameter of a mature plant from which the scion is derived.

According to some embodiments, the present invention provides a method for producing an engrafted plant, the method comprising: (i) providing at least two rootstocks and a scion; (ii) positioning a collar around the at least one scion; and (iii) grafting the scion to the at least two rootstocks.

According to some embodiments, the at least two rootstocks and the scion are derived from herbaceous plants.

According to additional aspect, the present invention provides an engrafted plant comprising a scion and a rootstock, wherein a collar is positioned around the rootstock.

According to an additional aspect, the present invention provides a method for producing an engrafted plant, the method comprising: (i) providing at least one rootstock and an intact plant; (ii) positioning a collar around the at least one rootstock; and (iii) grafting the at least one rootstock to the intact plant; to obtain an engrafted plant with a differential growth control.

The rootstock and the scion sources, as well as the collar are as described hereinabove.

According to certain embodiments, the rootstock with the collar supports the scion for a partial time of the growth. According to certain embodiments, the collar arrests the growth of the rootstock at a predetermined developmental stage, and therefore said rootstock is temporal. According to exemplary embodiment, following the growth arrest of the temporal rootstock, the remaining rootstock(s) maintains the grafted scion.

According to other embodiments, the rootstock with a collar supports the scion for the entire period of growth, wherein said rootstock supports the scion with differential intensities during the plant development.

According to another aspect, the present invention provides an engrafted plant comprising at least one scion and plurality of rootstocks wherein a collar is positioned around at least one rootstock.

According to certain embodiments, the rootstocks, the scion and the collar are as described hereinabove.

According to certain embodiments, the engrafted plant is of the Cucurbitaceae family According to exemplary embodiment, the scion is of a watermelon and the rootstock with the collar is of a gourd.

According to some embodiments, the present invention provides a method for producing an engrafted plant, the method comprising: (i) providing a rootstock of a watermelon and a rootstock of a gourd; (ii) positioning a collar around the gourd rootstock; and (iii) grafting the rootstocks together with one watermelon scion; to obtain an engrafted plant having a plurality of rootstocks providing differential growth control.

According to a yet another aspect, the present invention provides an engrafted plant comprising a continuous plant and at least one rootstock, wherein at least one rootstock has a collar.

According to certain embodiments, the grafted parts may be joined and held together by a ring or a plastic tape. According to exemplary embodiment, the grafted parts are held together with a Teflon tape. According to certain embodiments, the grafting region is sealed for keeping the moisture and preventing diseases. In exemplary embodiments, the grafting region is sealed with wax. The grafting should be kept in appropriate moisture and shading environment. It is to be explicitly understood that any other joining or sealing material and method as is known in the art can be used according to the teachings of the present invention.

The grafting point can be at any height of the plant. In some embodiments, the grafting point is below the cotyledons. In other embodiments, the grafting point is below the first true leaves.

According to certain embodiments, the grafting procedure is performed in a greenhouse in controlled temperature. In exemplary embodiments, the temperature is from about 17° C. to about 25° C. In some embodiments, the grafting is performed in controlled humidity. In exemplary embodiment, the humidity is from about 70% to about 99%. In another exemplary embodiment, the humidity is from about 90% to about 95%.

### Certain Embodiments of the Present Invention

The described figures illustrate several typical embodiments of the invention, which are further disclosed in the description of the present invention.

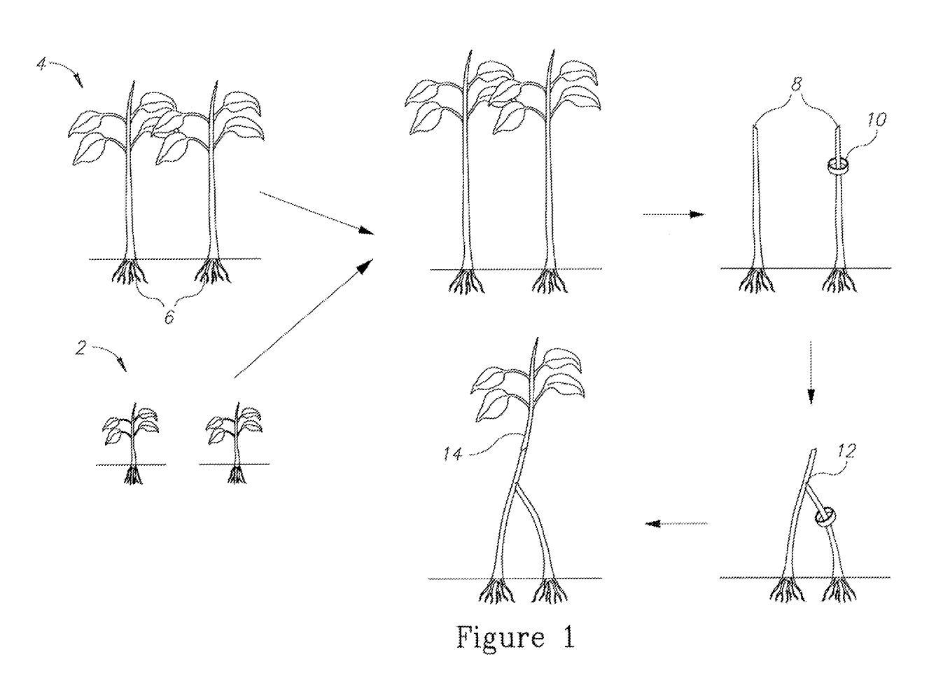

The present invention provides a grafting method, as shown in FIG. 1, comprising the steps of, first preparing the rootstocks (2 or 4). The plant sources for the rootstocks can be seeded, grown separately and then positioned next to the other 2, or readily planted 4 in a convenient distance 6 one from the other. The distance depends on the plants type and is such that they may be grafted to each other. According to certain embodiments, the distance should be from about 5 cm to about 20 cm. Next, the plants are cut 8 as to generate the rootstocks and a collar 10 is placed loosely around the designated rootstock. The rootstocks are then grafted to each other 12 and the scion 14 is grafted on the top of the rootstock. The collar may be positioned around any of the rootstocks.

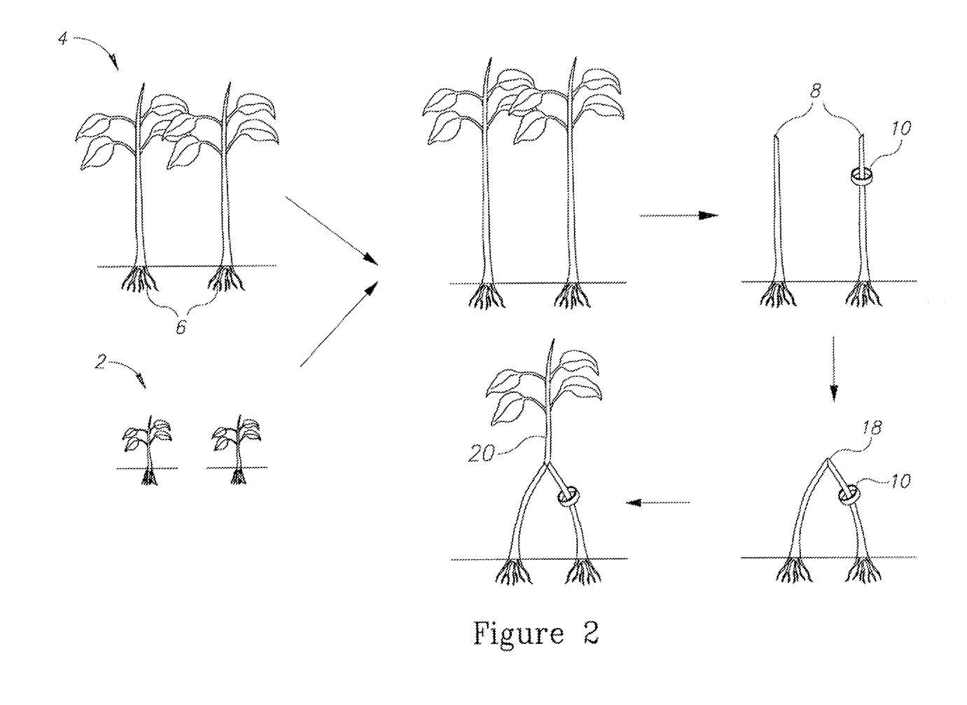

In alternate embodiments of the present invention, shown in FIG. 2, the two tops of the rootstocks are assembled. The plants are cut in transverse angles 18 in a way that enable their joining, while leaving space for the scion assembling. The scion 20 is then inserted to the space left at the top of the joint rootstocks.

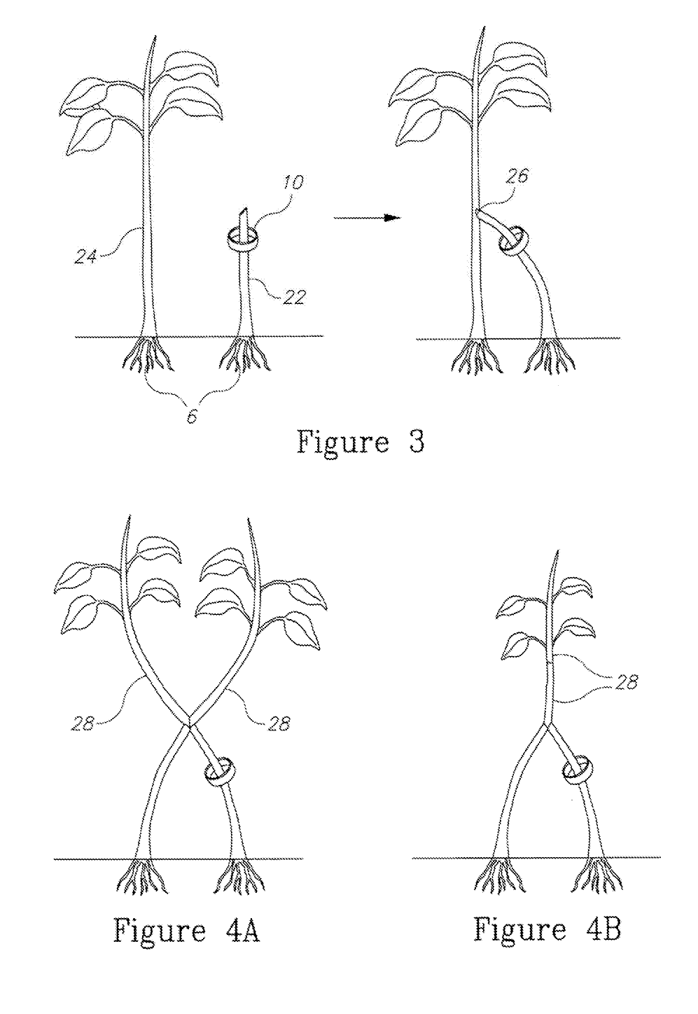

In alternate embodiments of the present invention, shown in FIG. 3, the method comprises the seeding and growing or planting of a grown plant that is the source for the rootstock 22 in a short distance from a whole plant 24, cutting the designated plant to generate the rootstock, and placing a collar 10 around the rootstock. Next, the rootstock is grafted 26 to the plant.

In alternate exemplary embodiments, shown in FIG. 4a and FIG. 4b, two scions 28 are grafted to the rootstocks. The two rootstocks can be grafted to the same connecting point (FIG. 4a) or one on top of the other (FIG. 4b).

The following examples are presented in order to more fully illustrate some embodiments of the invention. It should, in no way be construed, however, as limiting the broad scope of the invention. One skilled in the art can readily devise many variations and modifications of the principles disclosed herein without departing from the scope of the invention.

### EXAMPLES

### Example 1

### Yield of Engrafted Plants Comprising a Scion and Two Rootstocks

To examine the benefits conferred by two rootstocks upon scions, engrafted plants of tomatoes having two rootstocks were compared to non-grafted tomato plants. The effect of combination of two rootstocks originated from 3 different varieties of tomato was tested. Seeds of the Ikram (Zeraim Gedera), Arnold (Zeraim Gedera), and Maxifort (DE ruiter) varieties were seeded in a nursery in 3×3×3 cm pots. One month old seedlings were used for the engrafting experiments.

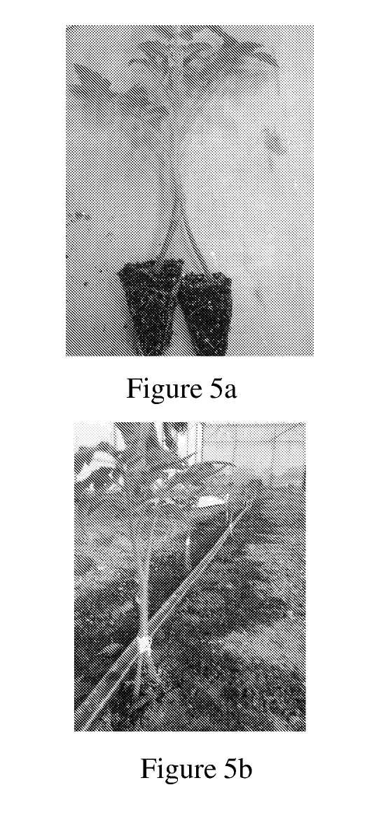

In a first assay, performed in the Arava region in Israel, Ikram variety was used as a scion. Rootstock varieties were as indicated in Table 1 below. The grafting procedure was performed as follows: the ends of the scion and one of the rootstocks were cut at 45 degrees angle and joined together. The connecting point was about 5 cm above ground. The second rootstock was cut at about 60 degrees angle and attached 1 cm above the first grafting point (FIG. 5a). The grafting points were fixed using a Teflon tape. The two rootstocks of the engrafted plant were placed back into adjacent pots. Next, the engrafted plants were transferred into controlled greenhouse having 95% humidity, 25° C. and light regime as known in the art. Three weeks later the plants were transferred to the field (FIG. 5b) and trellised as known in the art. The experiment included 4 replicates in a randomized order of planting. Plants were planted at 80 cm intervals (36 plants per tomato variety), and the Teflon was removed after 1 week. Control is Inkram variety plants that were planted at 60 cm intervals (56 plants). Fruit harvest was done after two and a half months (3 rounds of fruit-picking).

TABLE 1
A total yield of tomato fruits from plants of the
variety Ikram engrafted upon two rootstocks of
one of 3 different tomato varieties source.

| | | | | Total | |
|---|---|---|---|---|---|
| | | | Number | yield | Control- |
| | | | of | per | relative |
| Scion | Rootstock 1 | Rootstock 2 | plants | plant (kg) | percent |
| Ikram - non | — | — | 56 | 0.424 | 100 |
| grafted | | | | | |
| control | | | | | |
| Ikram | Ikram | Ikram | 36 | 0.457 | 107.78 |
| Ikram | Arnold | Arnold | 36 | 0.519 | 122.41 |
| Ikram | Maxifort | Maxifort | 36 | 0.585 | 137.97 |

The results in Table 1 show that multiple rootstocks increase the tomato yield as compared to non-grafted plants.

Additional experiment was performed in the Besor region in Israel. The grafting procedure was as described above. In this experiment, scions of the same variety of the rootstocks but with only a single rootstock were used as control.

TABLE 2
Average weight of tomato fruit produced by
engrafted plants with one or two rootstocks

| | | | Average tomato | relative |
|---|---|---|---|---|
| Scion | Rootstock 1 | Rootstock 2 | weight (gram) | percent* |
| Arnold | Arnold | — | 44.2 | 100 |
| Arnold | Arnold | Arnold | 49.4 | 111.76 |
| Maxifort | Maxifort | — | 40.5 | 100 |
| Maxifort | Maxifort | Maxifort | 81.1 | 200.25 |

*As compared to control.

The results demonstrate that a plurality of rootstocks of Arnold or Maxifort tomato varieties source increases the average weight of the tomato fruit.

### Example 2

### The Yield of Engrafted Plants That Comprise a Continuous Plant and a Supporting Rootstock

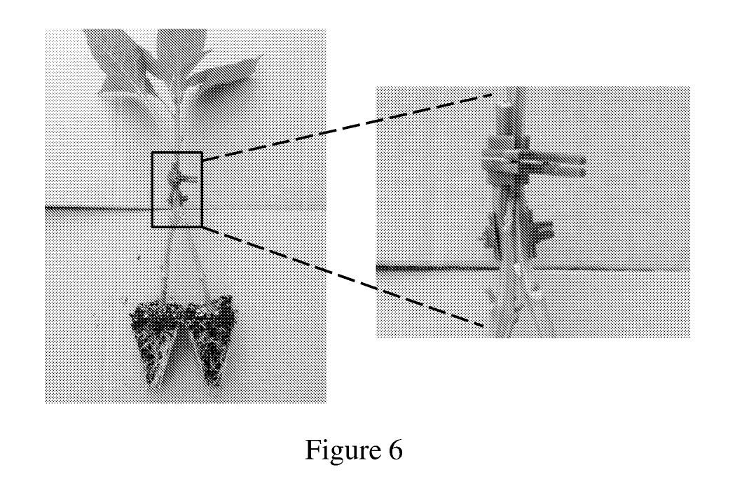

Next, the effect of two rootstocks on a single scion in an additional grafting method was examined FIG. 6 shows an engrafted plant having a supporting rootstock and the connecting clips (right panel, enlarged). The engrafted pepper plants were of the 7158 variety (Syngenta®). Peppers seeds were planted in trays having 3×3×3 cm pots. One month old seedlings were cut as to enable the joining of the vascular tissues. As exemplified in FIG. 6, one of the seedlings was left continuous from roots to the top and the upper part of the second one was cut off. The engrafted plants were transferred to a controlled greenhouse with 95% humidity and artificial light. The engrafted pepper plants were planted at 40 cm intervals.

TABLE 3
Number of fruit and total yield of engrafted pepper plants

| | Plants/plot | Number of | | |
|---|---|---|---|---|
| | trail | planting plots | Fruits per m² | Total yield |
| Control | 20 | 4 | 30.9 | 6.3 kg |
| Engrafted | 16 | 4 | 36 | 7.4 kg |
| plants | | | | |
| Engrafted | 20 | 4 | 25 | 5 |
| plants | | | | |

The results in Table 3 demonstrate that pepper plants at a density of 16 plants/plot trial, supported by additional rootstock, show increase in fruit number and total yield. Although having fewer plants per plot trial (20% less), the engrafted plants exhibit 17% increase in total yield (7.4 kg vs. 6.3 kg). In addition, the average number of fruits per m² was increased from 30.9 to 36.

The results also show that 20 engrafted plants per plot trial reduced total yield by 20% as compared to control. One reason for this reduction may be the result of high density of roots which negatively affect the plant growth and yield. The non grafted plants are planted in the maximum density known not to have negative effect on yield. A decrease in the density of the non-grafted pepper plants results in lower yield. A similar phenomenon is known in tomato, wherein engrafted plants enabled the reduction of plants per dunam (1000 m²) by half while maintaining the same yield.

### Example 3

### Engrafted Plant with a Scion That Benefits From the Use of Plurality of Rootstocks for a Predetermined Time

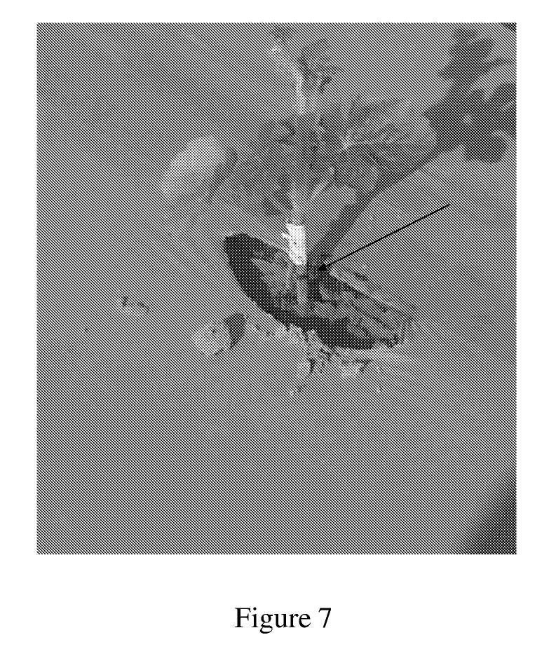

The watermelon ‘Ecstazy’ and ‘Shintoza’ varieties cannot grow in the adverse environmental conditions in the Arava region in Israel. The reasons are not entirely clear and apparently involve a variety of biotic and abiotic stresses such as heat, water quality and high amounts of the fusarium fungi in the soil. One solution is to support the watermelon plant by engrafting a rootstock of a gourd. The gourd rootstock provides vigor and resistance to the soil pathogens. However, as described hereinabove the grafting of the gourd rootstock have several negatives outcomes on the watermelon such as stiffed texture, decreased sugar level, and unfavorable taste. Another solution is to use a double rootstock system. The ‘Ecstazy’ watermelon was grafted on a rootstock of a gourd and a rootstock of an ‘Ecstazy’ watermelon. As exemplified in FIG. 7, a plastic collar was placed around the gourd rootstock at about 10 cm above ground. The collar diameter is about 10 mm. The collar is designed to suffocate and degenerate the rootstock at a predetermined stage. The collar is aimed to prevent the negative effect of the gourd rootstock during the ripening stage.

Overall, the results demonstrate that the use of multiple rootstocks may contribute to fruit size and total yield, which have great agricultural and economic benefits. In addition, as additional rootstocks may have negative effects in certain steps of development, a collar that limits the growth of one or more rootstocks can be highly useful.

1. A method for producing an engrafted plant, the method comprising: (i) providing a plurality of rootstocks; (ii) positioning a collar around at least one of the rootstocks; and (iii) grafting the plurality of rootstocks with at least one scion; to obtain an engrafted plant having a plurality of rootstocks each supporting growth for a predetermined period of growth.

2. The method of claim 1, wherein the collar arrests the growth of the rootstock at a predetermined developmental stage.

3. The method of claim 1, wherein the rootstock supports the scion throughout the growth period, wherein said rootstock supports the scion at differential intensities during the development of the engrafted plant.

4. The method of claim 1, wherein the collar is made of a material selected from the group consisting of a rigid material and a flexible material.

5-7. (canceled)

8. The method of claim 1, wherein the collar further comprises a growth inhibitor compound.

9-10. (canceled)

11. The method of claim 1, wherein the plurality of rootstocks and the scion are of herbaceous plants.

12. The method of claim 1, wherein the plurality of rootstocks and the scion are of plants of the same family.

13-15. (canceled)

16. A method for producing an engrafted plant, the method comprising: (i) providing at least one rootstock and an intact plant; (ii) positioning a collar around the at least one rootstock; and (iii) grafting the rootstock to the intact plant; to obtain an engrafted plant having a plurality of rootstocks supporting growth for a predetermined period of growth.

17. The method of claim 16, wherein the collar arrests the growth of the rootstock at a predetermined developmental stage.

18. The method of claim 16, wherein the collar supports the scion for the all period of growth, wherein said rootstock supports the scion with differential intensities during the plant development.

19. The method of claim 16, wherein the collar is made of a material selected from the group consisting of rigid material and flexible material.

20-22. (canceled)

23. The method of claim 16, wherein the collar further comprises a growth inhibitor compound.

24-25. (canceled)

26. The method of claim 16, wherein the plant and the at least one rootstock are of herbaceous plants.

27. The method of claim 16, wherein the plant and the at least one rootstock are of the same family.

28-30. (canceled)

31. An engrafted plant comprising a scion and a plurality of rootstocks, wherein a collar is positioned around at least one of said rootstocks.

32. The engrafted plant of claim 31, wherein the collar is made of a material selected from the group consisting of a rigid material and a flexible material.

33. (canceled)

34. The engrafted plant of claim 31, wherein the collar further comprises a growth inhibitor compound.

35-36. (canceled)

37. The engrafted plant of claim 31, wherein the two rootstocks and the scion are of herbaceous plants.

38. The engrafted plant of claim 31, wherein the two rootstocks are from the same family.

39-40. (canceled)

41. An engrafted plant comprising a plant and at least one rootstock wherein a collar is positioned around the at least one rootstock.

42-45. (canceled)
